(19) United States
(12) Patent Application Publication
(10) Pub. No.: US 2010/0331673 A1
(43) Pub. Date: Dec. 30, 2010

(54) SYSTEM AND METHOD FOR REDUCING PATIENT RISK OF ALLERGIC REACTION TO CONTRAST AGENTS OR MEDICAL MATERIAL

(76) Inventors: Michael Maschke, Lonnerstadt (DE); Gudrun Zahlmann, Neumarkt (DE); Martin Hahn, Marburg (DE); Michael Pringle, Brookline, MA (US)

Correspondence Address: BRINKS HOFER GILSON & LIONE, P.O. BOX 10395, CHICAGO, IL, 60610, US

(21) Appl. No.: 12/492,299

(22) Filed: Jun. 26, 2009

(51) Int. Cl. A61M 37/00; A61B 6/00

(52) U.S. Cl. 600/431; 604/66; 604/503

(57) ABSTRACT

A system and method for improving safety in medical procedures where a substance is introduced into a patient is described. An in-vitro diagnostic (IVD) device may be used to measure the concentration of an analyte representing a marker and compare the measured concentration with a predetermined set point so as to determine whether an adverse reaction to the substance may be anticipated. The test data may be obtained at the point-of-service or retrieved from a patient data base. The device for administering the substance is enabled when the comparison of the test data with the predetermined set point indicates that the procedure is expected to be performed with an acceptable risk of an adverse reaction.

### TECHNICAL FIELD

The present application relates to a system and method of improving patient safety when administering a substance during a procedure.

### BACKGROUND

Imaging procedures used in medical diagnosis or treatment may use a material, such as a contrast material, that is injected or otherwise dispensed into the patient being imaged. Contrast material may provide more detailed imaging information to a radiologist or other medical personnel responsible for analyzing the procedure results, by more clearly differentiating between tissue types, bodily structures, and the like. Such medical imaging procedures may use imaging modalities such as angiographic X-rays, computed tomography (CT), ultrasound (US), magnetic resonance imaging (MRI), or the like. The imaging modalities may provide either two dimensional or three-dimensional data of the particular patient, either as individual images or as a time series of images.

The term “contrast media”, as employed herein, refers to essentially any suitable type of media, as used in the medical arts, that is injected or dispensed into a patient and, in the context of a medical procedure for the purpose of highlighting selected areas of the individual's body while the individual is being imaged. In addition, the term “contrast media”, as employed herein, may also refer to other diagnostic or therapeutic agents for injection into individuals for diagnosis or treatment of syndromes. Contrast media may be injected into an individual's vasculature before or during a medical procedure by a dispensing device including, but not limited to, a power injector having an electronic controller.

Examples of dispensing devices used in medical imaging practice may be syringe-based power injectors (such as the E-Z-EM EMPOWER CT and EMPOWER CTA power injector systems, available from E-Z-M, Inc., Lake Success, N.Y.) that may include one or more syringes which may contain pre-loaded amounts of contrast media, and/or saline solution. In addition, such systems may be electronically controlled via electronic controllers that may be pre-programmed to administer a variety of contrast media, including other medical material, either arterially or intravenously in conjunction with medical imaging procedures or other medical procedures.

Some patients are, however, allergic to certain contrast media. The individual patient reaction may be just a metallic taste, a headache, or flushing of the skin, but occasionally more sever reactions such as anaphylactic shock occur. Reactions to non-ionic contrast agents are usually relatively harmless, but responses to ionic iodine-containing contrast agents may result in an anaphylactic-shock-like reaction. Such reactions may be life threatening and require an immediate reaction by the medical staff.

Moreover, during medical diagnosis and treatment, many tools, devices, tubes and catheters are placed on or introduced into a patient. Common materials encountered are copper, steel, gold, latex, ceramic, polyvinyl chloride (PVC), silicon and polyurethane in form of needles, tubes, catheters, staples, gloves, implants, and the like. Some patients are allergic to one or more of these materials. For healthy patients, allergic reactions to the materials may often be easily treated; however, a significant number of patients who are examined are sick and may have compromised immune systems, thus exacerbating the reaction.

One common allergy is to latex, which may be acquired during the course of a lifetime, or in the treatment of an illness by repeated exposure to latex protein when using, or in the presence of, sterile gloves, the use of condoms or diaphragms, or the like. Moreover, some of the substances used in manufacturing latex-based products are either allergens themselves, or facilitate the dispersal of other allergens.

The current clinical procedure is to ask the patient, his relatives or the referring doctor if the patient is allergic to contrast media or other materials which may be encountered in a hospital setting. The information may also be obtained from, or supplemented by, a computer-maintained data base having the patient medical history, paper records, or the like. However, such information sources have proved to be generally unreliable, as being mostly anecdotal. Further, where the patient might have had an allergic reaction, the person might have forgotten that the reaction happened, or did not associate the reaction with an allergen or remember the specific allergen involved, or the severity of the reaction was not understood. Moreover, the sensitivity to an allergen may increase substantially during a time interval between exposures thereto.

Where such an allergic reaction possibility is identified, there are a number of steps that may be taken to manage or mitigate the risk of a severe reaction to the substance being used. Such steps may include the selection of an another appropriate contrast medium or using other imaging or diagnostic procedures that do not depend on the contrast medium to which the patient is allergic, or not use contrast media, or using non-latex materials for gloves or other medical use.

In-vitro diagnostic (IVD) products are those reagents, instruments, and systems intended for use in diagnosis of disease or other conditions, including a determination of the state of health, in order to cure, mitigate, treat, or prevent disease or its sequelae. Such IVD products are intended for use in the collection, preparation, and examination of specimens taken from the human body.

Bio-markers and other IVD tests are becoming available to assist in patient diagnosis and treatment. A panel of markers may be needed in order to achieve clinically acceptable sensitivity and specificity for a particular purpose.

A blood analysis device such as “Lab on a Chip,” which is being developed by Siemens AG, may be used for determining blood values or certain genetic or molecular markers (see, for example, WO 00/56922, “Genetic Polymorphism and Polymorphic Pattern for Assessing Disease Status, and Compositions for Use Thereof”, and WO 00/23802, “Method for Measuring Cellular Adhesion” for gene tests and tests with molecular markers for stroke). See also, WO 2005/106024, entitled “Method and Assembly for DNA Isolation with Dry Reagents” and WO 2005/106023, entitled “PCR Process and Arrangement for DNA Amplification using Dry Reagents.”

The analytical chips may be credit-card sized systems into which a bodily fluid or a bodily tissue can be introduced, where a reaction takes place in the system and a result can be read out, normally with the help of an analytical chip holder into which the analytical chip is inserted.

Herein, the term “analyte” means a substance, chemical constituent, or component of a sample from a patient that is identified or quantified in an analytical procedure. For instance, in an immunoassay, the analyte may be a ligand or binder, while in blood glucose testing, the analyte is glucose.

An analyte itself may not be measured, but a measurable property of the analyte can be measured. For example, glucose may not be measured, but the concentration of glucose can be measured. In this example “glucose” would be the component of the sample and concentration is a property of the glucose component. Commonly, the term “property” is omitted, provided the omission does not lead to an ambiguity as to what property of the sample is being measured.

### SUMMARY

A system for improving patient safety is disclosed, including a treatment device having at least two operating states; and, an interface for accepting an operating state switching command. A first operating state inhibits the function of the treatment device, and the second operating state enables the function of the device. An operating state switching command is provided based on a comparison of an in-vitro diagnostic test data value with a pre-established set-point value.

In another aspect, a method increasing patient safety, includes the steps of determining a patient identity; selecting a procedure to be performed on the patient, where the procedure includes the dispensing of a substance, such as a contrast agent, into the patient. Where the patent has previously been tested for sensitivity to the substance, determining whether the test data value is below a safety set-point value; or, where the patient has not been tested for sensitivity to the substance, performing an in-vitro diagnostic (IVD) test for the sensitivity and determining whether the test data value is below a safety set-point value. Where the test data value is below the safety set-point value, a device for performing the procedure is enabled.

In yet another aspect, a computer program product, stored on a machine readable medium, includes instructions for configuring a computer to determine a patient identity, and accepts a user input selecting a procedure to be performed on the patient, the procedure including the dispensing of a substance by a device into the patient. For the selected procedure and substance, a substance-specific safety set-point value may be retrieved from a local memory, a therapy room memory or a data base. Where the patient data, which may be either local to the patient or retrieved from a data base, indicates that testing has previously been performed, the test data value is compared with the safety set-point value; or where the patient has not been tested for the sensitivity, an in-vitro diagnostic test for the sensitivity is requested, and resultant test data value is compared with the safety set-point value. When either of the comparisons indicates that the procedure may be safely performed, an enable command is transmitted to a device for administering the substance.

### BRIEF DESCRIPTION OF THE DRAWINGS

FIG. 1 is a block diagram of a therapy suite system including a capability for administering a substance to a patient; and

FIG. 2 shows steps in an example of a method for determining that a substance may be safely administered to a patient and enabling the administration of the substance.

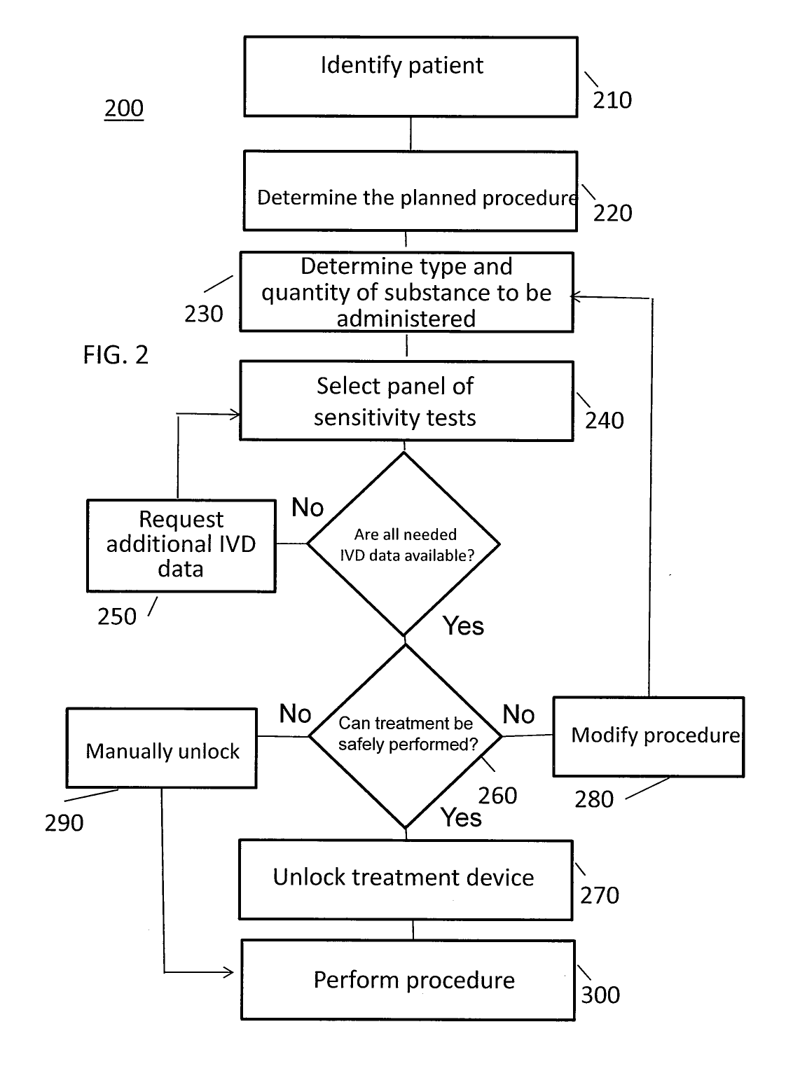

### DETAILED DESCRIPTION

Exemplary embodiments may be better understood with reference to the drawings. In the interest of clarity, not all the routine features of the implementations described herein are described. It will of course be appreciated that in the development of any such actual implementation, numerous implementation-specific decisions must be made to achieve a developers' specific goals, such as compliance with system and business related constraints and regulatory requirements, and that these goals and constraints will vary from one implementation to another.

The method and system described herein provides for assessment of the potential for an allergic-type reaction to the environment in which the patient is to be diagnosed or treated using a procedure. An IVD test is performed as a pre-requisite to the remainder of the procedure, and the test results read out and interpreted prior to proceeding. Where the potential of an allergic reaction is high, the material is changed, the procedure is modified, or pre- or post treatment steps are taken to mitigate or treat the expected reaction.

The medical equipment for the treatment or diagnostic procedure may be locked electronically so that the patient cannot be treated unless there exists a current or sufficiently recent set of test results in the hospital data base. The IVD testing may be performed in a patient room, the emergency room, an ambulance, or at the “point-of care.” The age of stored data that is acceptable may differ for different types of allergens. Only those IVD tests that may be appropriate for the planned procedure may be required to enable the medical equipment, authorize the patient to be moved to a treatment room, or the like.

The concerns associated with latex allergic reactions may be such that a patient should not be moved to an operating room, scheduled for a procedure, or the like, until a recent IVD result indicates a lack of sensitivity to latex-related allergens.

Where administering a contrast agent during an imaging procedure is contemplated, a “lock” may be applied to the power injector. Should a specific sensitivity to a contrast agent or other substance to be administered be indicated by the IVD test, another contrast agent may be selected, and this may cause the power injector to be unlocked. While the process of “locking” and “unlocking” is used with respect to a specific device in the description, other devices may be similarly controlled so that, for example, an X-ray procedure may not be enabled until a sensitivity of the patient to a substance to be administered during the procedure has been tested for and meets applicable safety criteria.

Some of the most commonly used contrast agents are the contrast agents most frequently associated with allergic-type reactions. Consequently, depending on the type and level of sensitivity, choices have to be made regarding as to whether to pre-treat the patient with reaction mitigating substances, to use a more expensive but more compatible contrast agent, or to change the procedure substantially to obtain the desired information without subjecting the patient to a substance likely to cause an adverse reaction.

Alternatively, as part of the protocol, a certain professional level of medical supervision may have the authority to override the “lock” so as to perform emergency treatment, for example. Moreover, alternatives to the IVD test result may be permitted where such a test is waived with informed consent by the patient.

The IVD analyser may be, for example, a point-of-care analyser: for example, the Siemens Clinitek Status (available from Siemens AG, Munich, Germany), or the Biosite Triage system (Biosite Incorporated, San Diego, Calif.) where the data output may provide specific procedure based instructions such as whether to proceed with imaging procedure, or to recommend modifying the contrast agent to a non-iodine-containing contrast agents to mitigate the possibility of anaphylactic-like reaction in a patient exhibiting signs of sensitivity to contrast agent.

In the modern hospital, the treatment rooms may be interconnected with the hospital data base of patient records, including records which may be part of a hospital, regional, or national data base. In such a circumstance, unneeded testing can be avoided by making reference to tests that have been previously performed, and whose results are considered timely. Whenever a next step in the treatment or diagnosis protocol is contemplated, the planning software may determine whether a particular IVD test result is a prerequisite, and “locks” the next step until the IVD test result is available, or the “lock” is otherwise properly overridden.

The medical equipment or movement order planning program may access an IVD test data base, by a communication medium, which may be a local or wide area network, wireless link, or the like. Alternatively a patient IVD card, which may have the current test results, may be accessed by a local card reader, or an IVD result which was produced by a central lab may be added to the data base. Whenever the appropriate test data is available and indicates that the procedure may be safely unlocked, the unlocking action is performed. The procedure may then proceed. Alternatively, if the IVD test result is not as yet available, or the result of the test counter-indicates the substance to be administered, or the procedure to be performed, the procedure remains locked. This may be done, for example, by electronically disabling the power injector.

When a device is unlocked for use, this may still mean that an operator may initiate the actual operation of the device, as there may be other preparations and safety procedures to be performed prior to the use of the device. However, in some instances, the power injector, for example, may be automatically activated and operated at a certain phase of the procedure, providing that it has been unlocked.

A specific panel of markers, such as a variety of immunoassays, may be developed using clinical or experimental studies to identify those markers most appropriate for predicting an allergic or other reaction to the substance to be administered. Since some of the markers may be appropriate for a plurality of screening applications, the storage of the results of previous IVD analyses in a data base of the patient medical history may be an efficient way of determining whether a new test is needed at the current stage of diagnosis or treatment. Some test results may be valid for an extended period of time, while others may desirably be repeated at frequent time intervals. For, example, an allergic reaction to latex-related proteins may intensify with time, and the progress of such sensitivity enhancement may not easily be extrapolated. A latex-specific IgE serology test (such as, the Siemens AlaSTAT, available from Siemens Medical Solutions Diagnostics, Los Angeles Calif.) or other suitable tests which may be subsequently developed, may be used to evaluate the presence and amount of antibodies specific to latex allergens (such as, Hev b 5 and Hev b 6), or other indicators.

A hospital or treatment room data processing facility may be configured to receive and process the data from the analytical (IVD) chip, or a central laboratory system may be a part of the medical device's environment. Alternatively, a plurality of such facilities, some of which may be specialized to particular IVD tests may be located more proximal to the specialty area where the results may be used. The test results may be communicated over a network and stored in a data base.

Analytical (IVD) chips are being continually developed with objectives such as reductions in analysis time, multiple factor and technique analysis of the same fluid sample, minimal external equipment requirements, and the like. As such, these chips may be used as screening devices for additional medical syndromes whose presence may influence the planning and selection of diagnostic or therapeutic techniques. When an IVD chip has the results stored thereon in a form that can be read out by an electronic card reading device, medical instrumentation may be programmed so that reading of an IVD card having appropriate test data may be needed in order to activate the device. For example, in a power injector used to administer a particular substance, the device is locked unless a patient IVD card is inserted, and the card has current test data indicating a lack of sensitivity to the substance. Alternatively, the device remains locked unless the patient data base has equivalent data for the patient, and the data base is accessible by the device, or by a device controller, which may be a computer that is part of the treatment suite.

An analytical chip reader, which may also include data analysis functions, can be integrated into the medical device, or connected to a data bus or a data network. That is, the IVD data may be read out by the reader, and the values of specific tests compared against set-point values computed for the patient, which may be dependent on the planned dose. In this manner, the IVD test results may be used for screening for more than one procedure, and the results interpreted in accordance with the specific planned procedure, as the threshold of sensitivity may vary for each patient and each procedure. Where pre-treatment with other substances is indicated to mitigate the possible allergic reaction, the patient data base may be checked to determine whether such treatment has been performed, and that an appropriate time has elapsed, before the treatment device is unlocked.

Alternatively, a doctor managing the treatment can access the data processing facility, and the data obtained by the IVD, and then, on the basis of the diagnosis, can make a decision about how to proceed in operating the medical device. Thus, the step of unlocking may be performed ether manually or automatically. A controller may send a command to the medical device, which command is received by the medical device, and decision logic, which may include a local controller, may act to place the device into either a first mode or a second mode, depending on whether or not the data which has been received, been processed, and fulfills a predefined criterion. As an example, a predetermined threshold value criteria or set point can be used

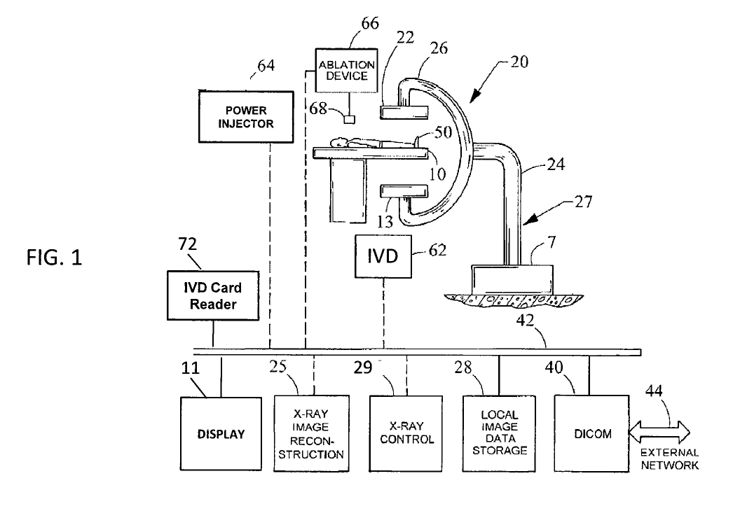

FIG. 1 shows a block diagram of an example of a system for the diagnosis and treatment of an illness by a use of a catheter to perform ablation therapy, further including a power injector for administering a contrast agent. Other embodiments of the system may include fewer than all of the devices, or functions, shown in FIG. 1. It will be understood by persons of skill in the art that the signal and data processing and system control is shown in an example, and that many other physical and logical arrangements of components such as computers, signal processors, memories, displays and user interfaces are equally possible to perform the same or similar functions. The particular arrangement shown is convenient for explaining the relationship of the elements and the functionality of the system. A similar treatment suite is described in US 2009/0069660, published on Mar. 12, 2009, by one of the present inventors, and commonly assigned, which is incorporated herein by reference.

An imaging modality, which may be a C-arm X-ray device 20 comprises a C-arm support 26 to which an X-ray source 22, which may include a diaphragm to limit the field of view, and an X-ray detector 13 may be mounted so as to face each other along a central axis of radiation. The C-arm 26 is mounted to a robotic device 27 comprising a mounting device 7, and one or more arms 24 which are articulated so as to be capable of positioning the C-arm X-ray device with respect to a patient support apparatus 10. A variety of other imaging modalities may be used, as is known, such as ultrasound imaging (US), magnetic resonance imaging (MRI), positron emission tomography (PET), single photon emission CT (SPECT). Other treatment devices such as photon and particle sources may also be used.

The C-arm X-ray device 20 is operable such that one or more projection X-ray images of a patient 50 may be obtained, and the images are reconstructed by any technique of processing for realizing radiographic or computed tomographic (CT-like) images. Alternatively, the C-arm X-ray device may be used to obtain real-time (fluoroscopic) images for guiding an interventional device, such as a catheter, which may be an ablation catheter 68. The power injector may be provided with one of a IVD card reader 72 or a bus connection to a network 42 so that the power injector 64 may unlocked by insertion an IVD card for the patient into a card reader that is part of the power injector 64, or by a IVD card reader 72 that is connected to the treatment suite data bus 42. The IVD card reader 72 may be integrated into the power injector 64 or connectable to the power injector directly or through a data bus 42 (as shown), or the like. An IVD device 62 may also be provided in the treatment room for point-of-service testing. Alternatively the data needed to unlock the power injector 64 may be retrieved from a patient data base located on a data base system maintained by the hospital or other entity.

The patient may be identified separately by the use of a wrist band, electronically readable tag, such as an RFID tag, or the like. The power injector 64 may be enabled after the appropriate patient and treatment information is verified, and may be initiated either manually or automatically, depending on the details of the procedure being performed.

The devices and functions shown in FIG. 1 are representative, but not inclusive, nor are all of the devices shown required. The individual units, devices, or functions may communicate with each other over cables or in a wireless manner, and the use of dashed lines for some of the data and control connections in is intended to suggest that alternative means of connectivity may be used.

In an aspect, the C-arm X-ray radiographic device 20 and the associated image processing 25 may produce angiographic or soft tissue computed tomographic images comparable to, for example, CT equipment, while permitting more convenient access to the patient 50 for ancillary equipment such as the catheter 68, power injector 64 and for treatment procedures. A separate processor 25 may be provided for this purpose, or the function may be combined with other processing functions.

Images reconstructed from the X-ray data may be stored in a non-volatile (persistent) storage device 28 for further use. The X-ray device 20 and the image processing attendant thereto may be controlled by a separate controller 29 or the function may be consolidated with the user interface and display 11.

The various devices may communicate with a DICOM (Digital Communication in Medicine) system 40 and with external devices over a local area network 42, and a hospital or regional data base over a network interface 44.

Some or all of the signal and data processing and data display may be located in the treatment room; however, equipment and functionality not directly related to the sensing or manipulating of the patient or the interventional device, may be remotely located. Such remote location may be facilitated by high speed data communications on local area networks, wide area networks, and the Internet. The signals representing the control information, data and images (generically “data”) may be transmitted by modulation of representations of the data on electromagnetic signals such as light waves, radio waves, or signals propagating on wired connections.

The system sensors, such as the IVD device 62 and the X-ray device 20 may thus be located remotely from the specialists making the diagnosis and for determining or administering the appropriate course of treatment. Of course, the specialists may be present with the patient at times as well.

A treatment suite, having at least a treatment device and an IVD device configured, which may not be collocated, to perform analyte measurements appropriate for the treatment to be performed may be used so to perform one or more of the methods described herein.

An example of a method 200 of mitigating the occurrence or severity of patient reaction to administered substances may include the acts of: identifying the patient to be diagnosed or treated (step 210); determining the planned procedure to be performed (step 220); and determining the type and quantity of a substance to be administered to the patient as a part of the procedure (step 230). Based on the type of substance, a panel of sensitivity tests is selected (step 240); for test data that is not current and is indicted as needed, the appropriate tests are administered by a in-vitro diagnostic (IVD) device (step 250). The results of the IVD testing are analyzed with respect to patient- and procedure-specific set points to determine if the patient may be safely treated (step 260). Providing that the results of the analysis indicate that the patient can be successfully treated, the treatment device is enabled (step 270). Where the test results indicated that the procedure should not be performed as planned, the test device remains locked until such time as the procedure is one of a) modified so as to meet the established safety requirements (step 280), or overridden by an authorized person (step 290). The procedure may then be performed (step 300).

Where a sequence of procedures is to be performed, the planning process may include evaluating the patient using IVD tests prior to commencing the first procedure, so that the IVD tests may be efficiently performed, the test data analyzed, and any procedure change made prior to commencement of the sequence of procedures. The IVD test data may be stored in the patient medical record so as to be available in each of the treatment suites through the hospital communications system, such as a LAN, when need to unlock various treatment devices. Alternatively, the IVD results may be stored on a patient IVD card that accompanies the patient to the treatment suites, and is read locally by a card reader and used either by a specific treatment device, or by a controller in the treatment suite to enable the appropriate treatment device.

It should be appreciated that as the use of IVD analyte panels becomes more widespread, other procedures may be performed and other syndromes may be treated using the methods described herein.

While the methods disclosed herein have been described and shown with reference to particular steps performed in a particular order, it will be understood that these steps may be combined, sub-divided, or reordered to from an equivalent method without departing from the teachings of the present invention. Accordingly, unless specifically indicated herein, the order and grouping of steps is not a limitation of the present invention.

The combination of hardware and software to accomplish the tasks described herein may be termed a platform or “therapy unit”. The instructions for implementing processes of the platform may be provided on computer-readable storage media or memories, such as a cache, buffer, FLASH, RAM, removable media, hard drive or other computer readable storage media. Computer readable storage media include various types of volatile and nonvolatile storage media.

The functions, acts or tasks may be independent of the particular type of instruction set, storage media, processor or processing strategy and may be performed by software, hardware, integrated circuits, firmware, micro code and the like, operating alone or in combination. Some aspects of the functions, acts, or tasks may be performed by dedicated hardware, or manually by an operator.

In an embodiment, the instructions may be stored on a removable media device for reading by local or remote systems. In other embodiments, the instructions may be stored in a remote location for transfer through a computer network, a local or wide area network, by wireless techniques, or over telephone lines. In yet other embodiments, the instructions are stored within a given computer, system, or device.

Where the term “data network”, “web” or “Internet” is used, the intent is to describe an internetworking environment, including both local (LAN) and wide area networks (WAN), where defined transmission protocols are used to facilitate communications between diverse, possibly geographically dispersed, entities. An example of such an environment is the world-wide-web (WWW) and the use of the TCP/IP data packet protocol, and the use of Ethernet or other known or later developed hardware and software protocols for some of the data paths.

Communications between the devices, systems and applications may be by the use of either wired or wireless connections. Wireless communication may include, audio, radio, lightwave or other technique not requiring a physical connection between a transmitting device and a corresponding receiving device.

While the communication is described as being from a transmitter to a receiver, this does not exclude the reverse path, and a wireless communications device may include both transmitting and receiving functions. There term “wireless communication” is understood to comprise the transmitting and receiving apparatus, including any antennas, and any modem used to encode or decode the data, speech, or the like, for transmission using electromagnetic waves.

Although only a few exemplary embodiments of this invention have been described in detail above, those skilled in the art will readily appreciate that many modifications are possible in the exemplary embodiments without materially departing from the novel teachings and advantages of the invention. Accordingly, all such modifications are intended to be included within the scope of this invention as defined in the following claims.

What is claimed is:

1. A system for improving patient safety, comprising:
a treatment device having at least two operating states; and
an interface for accepting an operating state switching command, wherein a first operating state inhibits the function of the device, and the second operating state enables the function of the device, and an operating state switching command is provided based on a comparison of an in-vitro diagnostic test data value with a pre-established set-point value.

2. The system of claim 1, wherein when the device is in the second operating state the device is caused to dispense a substance into the patient by a dispense command received through the interface.

3. The system of claim 1, wherein when the device is in the second operating state the device is caused to dispense a substance into the patient by activation of a switch.

4. The system of claim 1, further comprising an in-vitro diagnostic device (IVD).

5. The system of claim 4, wherein the IVD is a microfluidic device.

6. The system of claim 4, wherein the IVD performs an immunoassay.

7. The method of claim 4, wherein the IVD uses a lab-on-a-chip.

8. The method of claim 4, wherein the IVD uses a DNA micro-array.

9. The system of claim 1, wherein the interface includes a card reader for reading data from an in-vitro-diagnostic data card, and generating the operating state switching command based on a patient-specific set point value.

10. The system of claim 1, further comprising a computer communicating with a data base of patient data, the patient data including in-vitro diagnostic data.

11. The system of claim 10, wherein the set-point value is determined by a substance type to be dispensed into the patient.

12. The system of claim 11, wherein the set-point value is determined by a volume of the substance type to be dispensed into the patient.

13. A method of increasing patient safety, comprising the acts of:
determining a patient identity;
selecting a procedure to be performed on the patient, the procedure including the dispensing of a substance into the patient;
determining whether the patient has been tested for a sensitivity to the substance; and
where the patient has been tested for the sensitivity, determining whether a test data value is below a safety set-point value; or,
where the patient has not been tested for the sensitivity, performing an in-vitro diagnostic test for the sensitivity and determining whether a test data value is below a safety set-point value; and
enabling a device for performing the procedure if the test data value is below the safety set-point value.

14. The method of claim 13, further comprising storing patient test data on a computer data base.

15. The method of claim 13, further comprises the step of:
retrieving patient test data from the computer data base.

16. The method of claim 15, wherein the step of retrieving patient test data further comprises: obtaining the test data value from the computer data base over a local area network.

17. The method of claim 13, wherein in-vitro diagnostic test includes:
selecting a substance-specific diagnostic analyte type;
obtaining a sample of a bodily fluid; and
determining a value of the analyte type in the bodily fluid.

18. The method of claim 17, wherein the safety set-point value is selected based on the substance to be dispensed and the diagnostic analyte type

19. A computer program product, stored on a machine readable medium, comprising:
instructions for configuring a computer to:
determine an identity of a patient;
accept a user input selecting a procedure to be performed on the patient, the procedure including dispensing of a substance by a device into the patient;
retrieve a procedure and substance specific safety set-point value from a memory;
determine whether the patient has been tested for a sensitivity to the substance using patient data retrieved from a data base; and
where the patent has been tested for the sensitivity, determine whether the test data value is below the safety set point value; or,
where the patient has not been tested for the sensitivity, request an appropriate in-vitro diagnostic test, wait for results from the requested-vitro diagnostic test, and determine whether the test data value is below the safety set point value; and
transmit an enable command to the device if the test data value is below the safety set point value.

20. The computer program product of claim 18, wherein the set-point value is selected such that an in vitro test data value lower than the set point value is represents a safe situation.
